(19) United States
(12) Patent Application Publication
(10) Pub. No.: US 2009/0192385 A1
(43) Pub. Date: Jul. 30, 2009

(54) Method and system for virtual roadmap imaging

(76) Inventors: Oliver Meissner, Munich (DE); John C. Rauch, Savoy, IL (US)

Correspondence Address: BRINKS HOFER GILSON & LIONE, P.O. BOX 10395, CHICAGO, IL, 60610, US

(21) Appl. No.: 12/011,291

(22) Filed: Jan. 25, 2008

(51) Int. Cl. A61B 5/05

(52) U.S. Cl. 600/426

(57) ABSTRACT

A system and method of producing virtual roadmap image of a patient is described. CT-like image data may be obtained for a patient, with or without a contrast agent, and differenced so as to form a roadmap mask. The roadmap mask is in the form of a 2-dimensional digitally reconstructed radiograph from the same orientation as that of a fluoroscopic X-ray device which takes real-time images of the patient during treatment. The fluoroscopic image may be subtracted from the roadmap mask so as to more clearly visualize the position of a catheter introduced into the patient for treatment. When the orientation of the fluoroscopic image is changed during the course of treatment, the roadmap mask image for the corresponding orientation is used.

### TECHNICAL FIELD

The present application relates to a method and system of improving the medical treatment of patients using radiographic imaging.

### BACKGROUND

Fluoroscopy is an imaging technique where real-time projection X-ray images are obtained of a patient. Such images may be used to visualize the position of a treatment apparatus, such as a catheter, introduced into the patient body, or to visualize opacification resulting from administering a contrast agent, such as in an angiogram. The images are displayed to the user as they are acquired, allowing visualization of the interaction of the treatment apparatus with the anatomy, as in the case of interventional procedures. In interventional procedures the physician operates a treatment apparatus (e.g. catheter, guide-wire, or needle) disposed inside the patient, using fluoroscopy or another imaging modality to visualize the interaction of the device with the anatomy.

Roadmap fluoroscopy is of particular use in endovascular interventional procedures; that is, procedures that operate devices inside the arteries or veins of the patient. Roadmap fluoroscopy differs from standard fluoroscopy by highlighting the vessels in which the physician is operating devices for diagnosis or treatment. This is accomplished by combining a mask image with each X-ray image obtained by the imaging system to produce new “roadmap” fluoroscopic images. Depending on how the mask is constructed, different anatomy will be included or excluded in the roadmap fluoroscopy.

Currently, endovascular fluoroscopy roadmaps are produced from a rapid sequence of fluoroscopy images where a contrast agent is injected into a particular vessel or vessels of interest. The images of the short fluoroscopy sequence are then combined to create a single composite mask image of the vessels of interest. These images also contain all anatomy and devices that are visible under standard fluoroscopic examination. Additional scaling or processing of the image may be performed in converting the composite image into a roadmap mask image, and may vary depending on the manufacturer of the fluoroscopy system being employed.

Once the roadmap mask is created, the mask may be used until a new fluoroscopic working view orientation is required, or the patient anatomy position is altered (e.g. patient movement). If the procedure requires using roadmap fluoroscopy from a variety of viewing orientations, the fluoroscopic device must be adjusted to the new orientation and a new roadmap mask created. Creating additional roadmap masks, however, requires additional application of contrast agents and radiation to the patient, as well consideration of the time required to create a new roadmap mask.

### BRIEF SUMMARY

A system for imaging a patient is described, including an imaging device; and a digital storage device. The imaging device is configurable to collect imaging data suitable for producing CT-like images, and image data obtained with or without a contrast agent are processed to form a virtual roadmap mask image suitable for subtracting from a fluoroscopic image to produce a virtual roadmap image.

In another aspect, a method of imaging a patient is described, the method including the steps of: obtaining CT-like image data of a patient with or without administration of a contrast agent; computing digitally reconstructed radiographs for the collected image data at a projection corresponding to an orientation of a fluoroscopic apparatus; processing the digitally reconstructed radiograph to produce a virtual mask image. A fluoroscopic image of the patient from the same orientation as used to produce the virtual mask image is obtained and roadmap fluoroscopic images are obtained by, for example, subtracting the virtual mask image from the fluoroscopic image.

In yet another aspect, a computer-readable medium has instructions executable on a computer stored thereon, the instructions causing a computer system to receive image data from an imaging modality capable of collecting patient data suitable for production of CT-like image; compute a radiograph using the CT-like data in the same orientation as a fluoroscopic image of the patient; compute a virtual roadmap mask image by processing the computed radiograph; and, subtract the roadmap mask image from the fluoroscopic image data obtained for the same orientation.

### BRIEF DESCRIPTION OF THE DRAWINGS

FIG. 1 is a block diagram of a C-arm X-ray system for obtaining CT-like image data of a patient and for producing virtual roadmap mask images;

FIG. 2 is a simplified representation of the steps in a method of producing a virtual roadmap image, where (A) is a digitally reconstructed radiograph (DRR) obtained with no contrast media; (B) is the virtual roadmap mask for (A); (C) is the fluoroscopic image acquired by the imaging system; (D) is the virtual roadmap image resulting from the difference of the (C) and (B); (E) is a DRR obtained using a contrast media; (F) is the virtual roadmap mask for (E); (G) is the fluoroscopic image acquired by the imaging system; and (H) is the virtual roadmap image resulting from the difference of (G) and (F);

FIG. 3 is a flow chart of the method; and

FIG. 4 is a flow chart of the step in the method of FIG. 3 of producing a virtual roadmap mask.

### DETAILED DESCRIPTION

Exemplary embodiments may be better understood with reference to the drawings. Like numbered elements in the same or different drawings perform equivalent functions.

In the interest of clarity, not all the routine features of the examples herein are described. It will of course be appreciated that in the development of any such actual implementation, numerous implementation-specific decisions must be made to achieve a developers'specific goals, such as consideration of system and business related constraints, and that these goals will vary from one implementation to another.

The examples of diseases, syndromes, conditions, and the like, and the types of examination and treatment protocols described herein are by way of example, and are not meant to suggest that the method and apparatus is limited to those named, or the equivalents thereof. As the medical arts are continually advancing, the use of the methods and apparatus described herein may be expected to encompass a broader scope in the diagnosis and treatment of patients.

An X-ray imaging modality may comprise an X-ray tube, high-voltage power supply, radiation aperture, X-ray detector, digital imaging system, system controller, as well as user control and display units. The X-ray detectors may be amorphous Selenium (a-Se), PbI2, CdTe or HgI2 detectors using direct detection and TFT technology, or indirect detectors as is known in the art, or may be subsequently be developed, to provide high resolution, high-dynamic-range real-time X-ray detection. The X-ray detector may be disposed diametrically opposed to the X-ray source and such that the plane of the detector is perpendicular to the axis of the X-ray source. This orientation may, for example, be maintained by attaching the X-ray source and X-ray detector to a C-arm, a U-arm or the like. The C-arm may be mounted to a robot so as to permit the X-ray source and detector to be oriented with respect to the patient.

The C-arm X-ray device is provided with an X-ray source and an X-ray detector, and may be operated to obtain fluoroscopic images, data suitable for the production of 2D, images, or computed tomography (CT)-like 3D images. The images may be synchronized with the cardiac cycle.

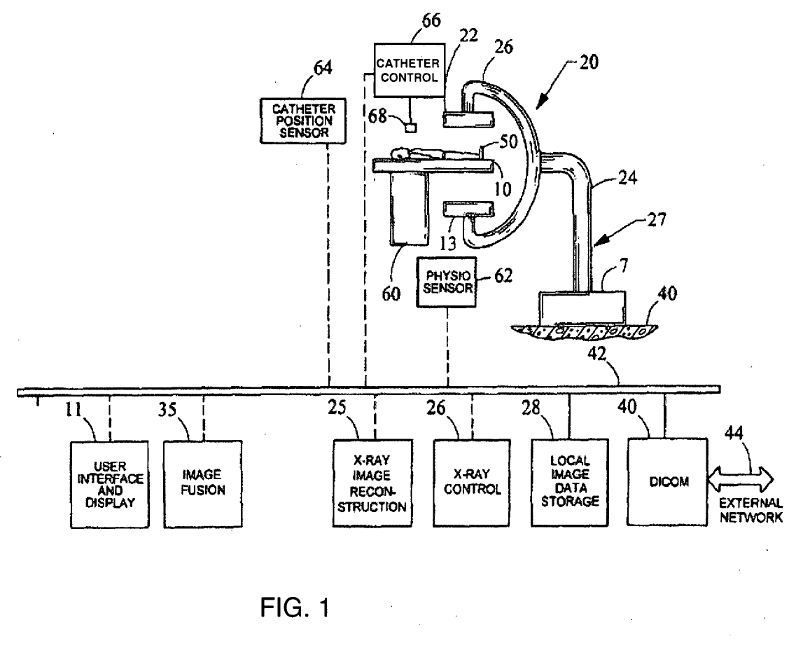

The use of the term CT-like data or CT-like images is understood to encompass data and images obtained by a CT X-ray device or other tomographic imager. FIG. 1 shows a block diagram of an example of a system for the diagnosis and treatment of an illness by a use of a catheter. Other embodiments of the system may include more than, or fewer, than all of the devices, or functions, shown in FIG. 1.

The data processing and system control is shown as an example, and many other physical and logical arrangements of components such as computers, signal processors, memories, displays and user interfaces are equally possible to perform the same or similar functions. The particular arrangement shown is convenient for explaining the functionality of the system.

The C-arm X-ray device 20 may comprise a C-arm support 26 to which an X-ray source 22, which may include a diaphragm to limit the field of view, and an X-ray detector 13 may be mounted so as to face each other about an axis of rotation. The C-arm 26 may be mounted to a robotic device 27 comprising a mounting device 7, and one or more arms 24 which are articulated so as to be capable of positioning the C-arm X-ray device with respect to a patient support apparatus 10. The robotic device 27 may be controlled by a control unit 11, which may send commands causing a motive device (not shown) to move the arms 24. The motive device may be a motor or a hydraulic mechanism. The mounting device may be mounted to a floor 40 as shown, to a ceiling or to a wall, and may be capable of moving in longitudinal and transverse directions with respect to the mounting surface.

The C-arm X-ray device 20 is rotatable such that a sequence of projection X-ray images may be obtained by an X-ray detector 13 positioned on an opposite side of the patient from the X-ray source 22, and the images may be reconstructed by any technique of processing for realizing computed tomographic (CT)-like, 2-D, or real-time fluoroscopic images. A patient 50 may be positioned on a patient support apparatus 10. The patient support apparatus 10 may be a stretcher, gurney or the like and may be attached to a robot 60. The patient support apparatus 10 may also be attached to a fixed support or adapted to be removably attached to the robot. Aspects of the patient support apparatus 10 may be manipulable by the robot 60. Additional, different, or fewer components may be provided.

The devices and functions shown are representative, but not inclusive. The individual units, devices, or functions may communicate with each other over cables or in a wireless manner, and the use of dashed lines of different types for some of the connections in FIG. 1 is intended to suggest that alternative means of connectivity may be used.

The C-arm X-ray radiographic device 20 and the associated image processing 25 may produce angiographic and computed tomographic images comparable to, for example, CT equipment, while permitting more convenient access to the patient for ancillary equipment and treatment procedures. A separate processor 25 may be provided for this purpose, or the function may be combined with other processing functions. The various devices may communicate with a DICOM (Digital Communications in Medicine) system 40 and with external devices over a network interface 44, so as to store and retrieve image and other patient data.

Images reconstructed from the X-ray data may be stored in a non-volatile (persistent) storage device 28 for further use. The X-ray device 20 and the image processing attendant thereto may be controlled by a separate controller 26 or the function may be consolidated with the user interface and display 11.

The X-ray images may be obtained with or without various contrast agents that are appropriate to the imaging technology and diagnosis protocol being used.

Additionally, a physiological sensor 62, which may be an electrocardiograph (ECG), a respiration sensor, or the like, may be used to monitor the patient 50 so as to enable selection of images that represent a particular portion of a cardiac or respiratory cycle as a means of minimizing motion artifacts in the images.

Where the terms “CT-like data” or “CT-like” images are used, this is meant to include data obtained from an apparatus such as a conventional closed-type CT device, from a C-arm X-ray device, or the like, or from any technology that produces data characterizing a property of the internal volumetric structure of a patient. This could, for example, include acoustic tomography or magnetic resonance imaging (MRI).

Where the term “subtracted” is used in conjunction with image manipulation, this is not meant to exclude addition, with or without gray scale modification, transformation, or adjustment. Images may be represented as either positive or negative images.

The treatment device 66 may be a catheter 68 which is introduced into the body of the patient 50 and guided to the treatment site by images obtained by the C-arm X-ray, or other sensor, such as a catheter position sensor 64. The catheter position sensor may use other than photon radiation, and electromagnetic, magnetic and acoustical position sensors are known.

A catheter locating system (for example, U.S. Pat. No. 5,042,486, “Catheter Locatable with Non-Ionizing Field and Method for Locating Same”,) can be integrated into the system. The catheter may be provided with position sensors, such as electromagnetic sensors or magnetic sensors. Thus the tip of the catheter, in particular, can be detected without emitting continuous X-rays and the motion thereof can be followed and displayed with respect to a previously obtained image by adding the catheter position to the images synthetically.

In another alternative, an Acunav catheter (ultrasound catheter) can be used in addition to the X-ray images, in order to use 3D ultrasound images in real time for guiding the catheter. (see, for example, U.S. Pat. No. 6,923,768, “Method and Apparatus for Acquiring and Displaying a Medical Instrument Introduced into a Cavity Organ of a Patient to be Examined or Treated”).

Apart from the sensors and positioning capabilities, the imaging, data processing, and controlling equipment may be located within the treatment room or remotely, and the remotely-located equipment may be connected to the treatment room by a telecommunications network. Aspects of the diagnosis and treatment may be performed without personnel, except for the patient, being present in any of the local treatment rooms.

The X-ray imaging device may be operated by rotating the C-arm such that the opposed X-ray source and X-ray detector traverse an angular range of at least about 180 degrees about an axis perpendicular to the plane of the C-arm. A 3D image may be reconstructed from the detected X-ray data, or 2D images or fluoroscopic images may be reconstructed in various image planes. The algorithmic and measurement aspects of computed tomography images are being improved, and the processing of the images obtained by the imaging devices are expected to continue to improve in resolution and dynamic range, speed, and in reduction of the X-ray dosage.

The term “X-ray” is used to describe any device that uses ionizing radiation to obtain data regarding the opacity of a path through a patient, or part thereof, regardless of the wavelength or source of the radiation used. Where the term “catheter” is used, it is intended to represent any treatment apparatus introduced into the patient's body, and may also include contrast agents introduced intra-operatively to visualize the results of a procedure.

The positioning of a catheter inside a patient, and the manipulation of the catheter position to administer treatment is facilitated by the use of real-time fluoroscopic images of the patient. Alternatively, the position of the catheter or other apparatus may be measured by acoustic or magnetic means, and superimposed on a fluoroscopic or 2-D X-ray image, and the image may be a previously obtained image, or based on previously acquired data.

A fluoroscopic roadmap image is produced by obtaining a fluoroscopic image data set of the patient in the same position as treatment will be administered; administering a contrast agent so as to visualize the structure to be treated, and making a composite image of a series of images taken during the administration of the contrast agent so as to produce a mask image.

The radiographic image may be formed by the detection of X rays that have been exponentially attenuated in passing through the body. The attenuation of X-rays in passing through the anatomy of a patient is a loss per unit length, and when integrated along the path of the ray between the source and the detector, is exponentially related to the spatially varying absorptive properties of different types of tissue, including bone. This attenuation is commonly measured in Hounsfield units.

The brightness of the objects in the digitally subtracted angiographic image (e.g., the vessels with contrast material) is not substantially affected by the brightness (density) of the underlying tissues in the non-subtracted images. As the X-ray beam is not mono-energetic, the subtraction is not perfect; and there may still be a slight variation of vessel brightness that is dependent on the attenuation of the underlying tissue. However, the image details of the patient's anatomy may be substantially suppressed.

Road mapping fluoroscopy is a useful technique for the placement of catheters and wires in complex and small vasculature, but may be also used to visualize any material introduced into the patient's body. A DSA (digital subtraction angiography) sequence may be performed, and the image with maximum vessel opacification identified; this image may be selected as the road map mask. The road map mask may be subtracted from subsequent fluoroscopic images to produce real-time subtracted fluoroscopic images overlaid on a static image of the vasculature.

Here, either the radiograph reconstructed from CT-like data where contrast is administered or not administered may be used as a virtual road map mask. As used herein, the term “virtual” is intended to suggest that at least a portion of the resultant image was computed from pre-stored data or that at least some of the data were obtained in an image plane differing from the image plane in which the image is now being used.

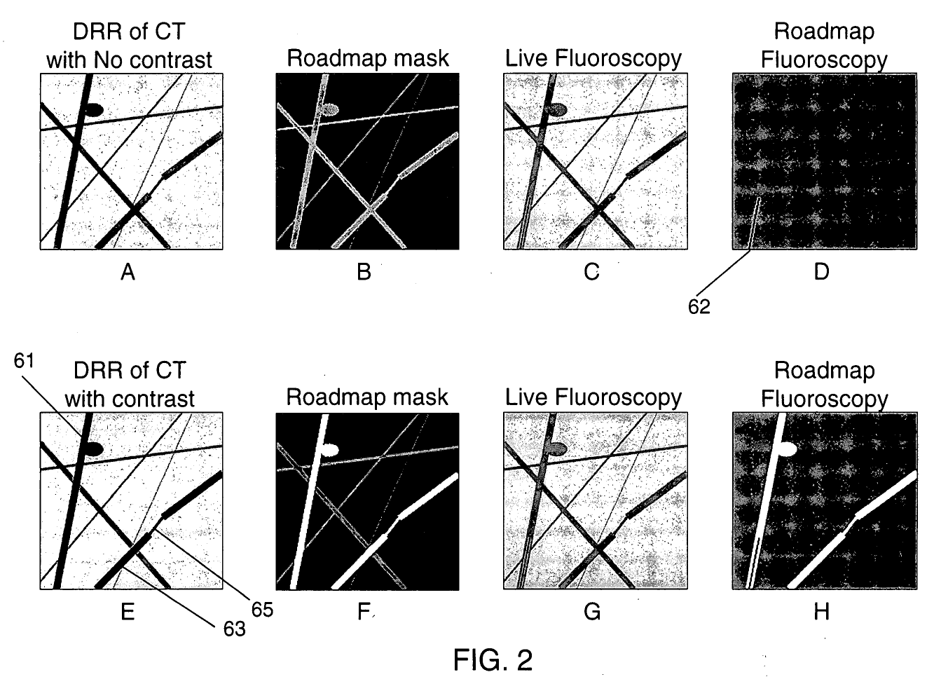

FIG. 2 is a schematic representation of the preparation and use of a virtual roadmap mask. A 2-D image of the working area is taken using an X-ray apparatus to produce a radiographic image as shown in FIG. 2A. Such an image may also be computed as a digitally reconstructed radiograph (DRR) from previously obtained CT-like image data, where the image is reconstructed using the orientation selected for the fluoroscopic view. The DRR may be converted into a virtual roadmap by suitable manipulation of the gray-scale values. In FIG. 2B, the gray scale is inverted. FIG. 2C shows a fluoroscopic image taken from the same orientation as the virtual roadmap mask, and FIG. 2D shows the result of subtracting the image of FIG. 2C from that of FIG. 2B, which suppresses the details of the patient's anatomy. This reveals the presence of catheter 62 which as been introduced into the patient.

From the same vantage point, a 2-D image of the working area is produced (FIG. 2E) using a contrast agent introduced in to the vessel or organ to be visualized. In this view, the contrast agent has resulted in enhancement of two vessels, which may have a common point (not shown) from which the contrast agent has propagated. The darker shading indicates that the attenuation through the vessel has been increased which respect to that where no contrast agent is present. In practice, additional details of the morphology of the vessels may be seen when using contrast agents. FIG. 2F shows a roadmap mask image produced from the digitally reconstructed radiograph (DRR), by performing such image processing as may be needed to be compatible with the fluoroscopic imaging system on which the mask is to be used (e.g., inverting intensity values). The live fluoroscopy image shown in FIG. 2G may be the same as shown in FIG. 2C, and when the images of FIG. 2F and 2G are subtracted, the contrast-enhanced vasculature is seen, as well as the location of the catheter with respect thereto. This enables the position of the catheter 62 to be better visualized with respect to the anatomical structures that were subject to previous contrast enhancement. Here, an aneurism 61 and a stenosis 65 in a blood vessel 63 may be seen. After treatment, another fluoroscopic image may also be taken using contrast material and may use the virtual roadmap mask, so as to determine the changes in vessel interior diameter, or the state of an aneurism.

The term “virtual roadmap mask” is used herein to describe a computed 2-dimensional radiograph, produced from CT-like data, so as to positioned by the computation to be from the same orientation as fluoroscopic data of the same patient, where the virtual roadmap mask and the fluoroscopic data are used to produce a composite image, which may be termed a “virtual roadmap image”. In an aspect, the position of a catheter may as determined by a technology such as acoustic imaging may be used in place of, or to complement, the data obtained by fluoroscopy, and be superimposed on the virtual roadmap mask to produce a virtual roadmap image.

During the procedure, fluoroscopic images may be desirable from more than one vantage point (working view, orientation) so as to better visualize the relationship of the catheter to the patient, and fluoroscopic images may be taken from other orientations, or where the patient has been moved. The virtual road map produced for the original orientation is inappropriate for the new orientation, as the road map is generally no longer congruent with the structure that it represents. A new road mask may need to be produced, involving further administration of contrast agent and X-ray exposure.

Providing that CT-like images of the patient, for the region of interest, have been previously obtained and that the CT data set includes data obtained during a period where a contrast agent has been administered, a “virtual roadmap mask” may be produced. A virtual roadmap mask is created using Digitally Reconstructed Radiograph (DRR) images of the patient computed from the CT dataset of the same patient.

The DRR is a synthesized fluoroscopic (2-D radiograph) image of a patient as if a fluoroscopic image had been taken from a specific orientation of the X-ray apparatus. In effect, the ray paths from the X-ray source to the detector are integrated through the attenuation densities of the body determined by the CT volumetric imaging process, so as to compute the total X-ray attenuation for each linear ray path. DRR images therefore closely approximate real-time fluoroscopy images and are used in some 2D to 3D image registration algorithms, where DRR images are iteratively computed from slightly different orientations, and compared to an actual fluoroscopy image.

The CT-like data may be used to compute a synthesized 2-dimensional image (DRR) from a specified orientation. The image produced by DRR may therefore be produced with respect to a differing orientation from the CT-like data collection axis of rotation, as the tomographic process results in a volumetric measurement of tissue absorption characteristics for each resolved voxel. A DRR may thus be produced from for an arbitrary location from the stored data, and the orientation may be selected to correspond to a specified orientation of the X-ray apparatus for a current fluoroscopic image. A corresponding virtual roadmap may be produced. Such views during a procedure may be termed “working views”.

The term DRR may therefore correspond to a 2-D image for a particular orientation of the X-ray apparatus, and be substantially the same as a real-time X-ray image (fluoroscopic image) taken at the same orientation.

Where the CT-like data has been previously obtained, the CT-like image data set may retrieved from a data base and may be used to produce a DRR from an orientation with respect to the patient that corresponds to the orientation of the fluoroscopic device with respect to the patient. This may be done without further administration of contrast agent, or exposure of the patient to ionizing radiation, as the data suitable for this reconstruction has already been obtained as part of the CT-like data set. In an aspect, the DRR may be processed by, for example, gray-scale inversion, so as to form a virtual roadmap mask corresponding to the fluoroscopy viewing orientation. The virtual roadmap mask may be subtracted from the fluoroscopic image so as to produce a virtual roadmap fluoroscopic image. This virtual roadmap image may then be used in conjunction with real-time fluoroscopic images having catheter images embedded therein, or other catheter location data, taken from the specified viewing orientation.

Since the data set of the CT-like image provides volumetric data on the tissue and contrast agent opacities, the fluoroscopic image produced as a DRR or the resultant virtual roadmap mask image is not limited to the angular space orthogonal to the patient axis, and just as CT slices of the body may be produced off of the principal axes of the patient, the patient may be viewed at an oblique angle and a corresponding 2-D radiograph computed or virtual roadmap mask image produced.

The CT data set should be taken with the patient in a known position (e.g., prone, supine) and the best quality image is likely when the data is obtained as close as possible in time to the use thereof.

Flat detector fluoroscopic imaging systems, such as the Siemens C-arm “Axiom” (available from Siemens AG, Munich, Germany) are capable of acquiring CT-like data sets of the subjects, and it is becoming increasingly common to acquire 3D or CT-like datasets with contrast injected into specific vessels to enhance diagnosis or enable planning for interventions. The acquisition of the CT-like data set to produce the virtual roadmap mask is not expected to be a burden on the workflow for standard diagnostic and interventional procedures. Rather, it may reduce the need for multiple administrations of contrast agents.

When the CT-like dataset is acquired using the fluoroscopy system where virtual roadmap mask is to be employed, there is an implicit registration of the CT-like dataset and the fluoroscopic imaging system, or other associated catheter location system, as the patient is in the same position for the CT-like dataset acquisition as for fluoroscopy and the geometries of these two acquisitions are intrinsic to the design of the imaging system.

A method of using a virtual roadmap in the treatment of a patient is described. A patient is positioned with respect to an X-ray apparatus, the apparatus being capable of obtaining fluoroscopic images and obtaining image data from which CT-like images may be produced. Alternatively, a CT-like data set previously obtained may be imported from a medical data base such as DICOM (Digital Communications in Medicine) and registered with respect to the patient position.

The method may include obtaining the image data base, producing the virtual roadmap mask, and using the virtual roadmap during a treatment procedure. Some of the steps are performed automatically by the system, and some of the steps may be performed by medical personnel, and the description of a step as automatic or manual is not intended to preclude the other.

The virtual roadmap mask data source is identified. A CT or CT-like data set may be identified as a data set for the procedure and to be used for the calculation of DRR images for producing virtual roadmap masks. If an appropriate data set cannot be identified, such a data set is obtained by positioning a patient with respect to the X-ray apparatus, and using the X-ray apparatus to obtain data suitable for producing CT-like images. As appropriate, contrast material may be administered so as to provide data relating to opacified regions.

The virtual roadmap mask image may be produced by computing a DRR image for each imaging plane of the imaging system (e.g., one DRR for a single plane system or two DRRs for a biplane system) for the data set having the contrast agent. Where the DRR is produced using data representing the administration of a contrast agent, the virtual roadmap mask may then be subtracted from the real-time fluoroscopic image so as to display the catheter. A virtual roadmap mask image may be produced for each imaging plane, and for any subsequently selected imaging plane.

The virtual roadmap also may be produced by the steps of computing a DRR image for each imaging orientation of the imaging system (e.g., one DRR for a single plane system or two DRRs for a biplane system). The corresponding virtual roadmap mask images may be subtracted from fluoroscopic images obtained in each of the imaging orientations to produce virtual roadmap images. Where the two imaging planes are substantially orthogonal, and are either available simultaneously or in rapid succession, the virtual roadmap images thus produced may aid in the location of the catheter within an anatomical structure.

The position of the catheter may be determined by other means, such as acoustic and magnetic means previously described, and superimposed on the digitally subtracted mask image so as to produce a virtual roadmap showing the catheter position.

Where the apparatus and method is being used in the treatment of a patient, the medical professional may wish to view the anatomy and catheter from another orientation. The system may be commanded through a computer terminal interface of haptic device so as to more the imaging plane to another orientation. As such, the current road map image will not longer be congruent with the fluoroscopic image. Often, the user will adjust the orientation of the X-ray apparatus using the fluoroscopic images until the view presented appears satisfactory for the next stage of treatment or evaluation. When the system has stopped moving, or the user requests a new virtual roadmap mask, the system will retrieve the CT-like image data from the data base, which may be a local or remote data base, and compute a new virtual roadmap mask image corresponding to the new orientation.

When the user begins to use the fluoroscopic images, the virtual roadmap mask may be subtracted from the fluoroscopic images and the resultant image displayed. As multiple displayed images are often used, the fluoroscopic image and the virtual roadmap image may be simultaneously displayed. This process may be repeated as often as is necessary in the performance of the medical procedure.

Although the process has been described as retrieving the CT-data from a data base, the retrieval of the data and the computation for a DRR and virtual roadmap mask may be performed at a remote location, and only the resultant DRR or virtual roadmap mask transmitted to the computer associated with the local processing and display function. That is, nothing herein is intended to limit the location at which data processing, storage, or display is performed. Such specific locations are merely convenient for descriptive purposes. The use of the virtual roadmap procedure may be discontinued at any time and conventional fluoroscopy used.

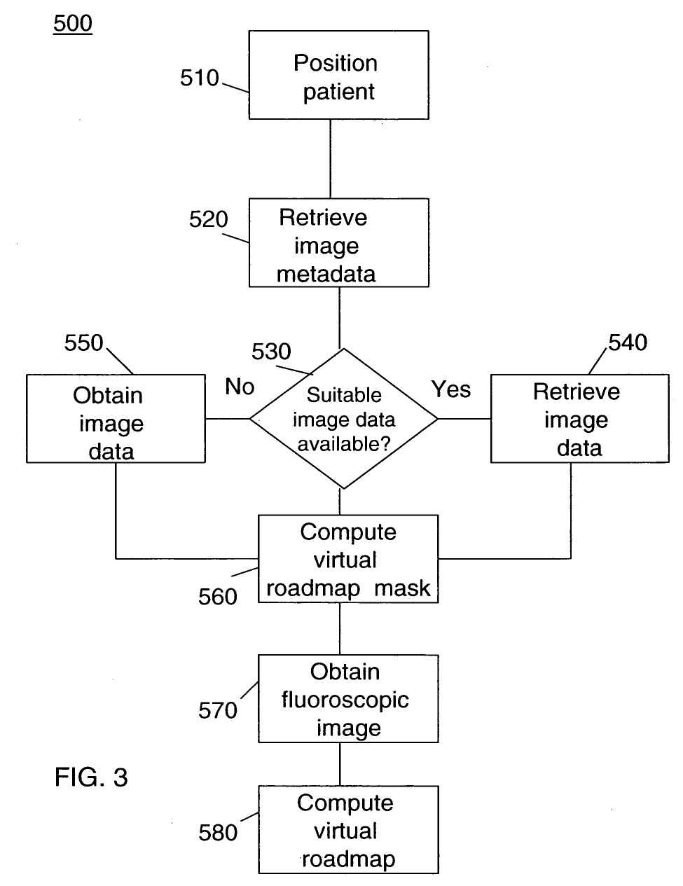

In an example of a method of producing a virtual roadmap, the method 500 as shown in FIG. 3 begins with positioning the patient with respect to the fluoroscopic imaging device (step 510). The user, who may be a physician or technician may attempt to retrieve image data that has previously been taken for the patient and which may be suitable for use. The suitability of such data may be determined using image metadata, or the image data itself may be retrieved and viewed. The data needs to at least represent the region of the patient body in which the diagnosis or treatment procedure is intended to be performed. If such images are available (step 530), then the image data is retrieved from the data base in which it has been stored (step 540). The image data is in a CT-like form and a digitally reconstructed radiograph (DRR) is computed to yield a 2-D radiograph, corresponding to a fluoroscopic image which would be obtained with the same orientation of the X-ray device with respect to the patient. The orientation that is used to produce the DRR may be adjusted using image matching techniques so as to match a fluoroscopic image taken of the patient in real time.

The computation of the DRR may be performed locally using data stored locally or remotely. Alternatively, the orientation parameters may be transmitted over a network to a remote location where the CT-like data is stored, and the DRR may be computed remotely and then transmitted to the local site where the procedure is being performed.

Where there is no suitable stored data, the X-ray apparatus, which may be a C-arm X-ray device, may be used to obtain the CT-like image data (step 550). This data may be obtained with or without administration of contrast media. The steps of preparing the patient for this procedure are not shown.

The virtual roadmap mask is similar to a conventionally acquired roadmap mask image and may be computed from the DRR by, for example, gray scale inversion. However, the produced virtual roadmap images may be for any orientation of the fluoroscopic X-ray device, including orientations that may be oblique to the axis of rotation about which the CT-like data was obtained. This is possible as the CT-like image data set is suitable for the computation of DRR images, and represents the volumetric specific absorption properties of the patient. The virtual roadmap mask image may be subtracted from the acquired fluoroscopic images taken at the specific orientation to produce virtual roadmap fluoroscopic images. These images suppress the overall patient anatomy image features and highlight devices inserted into the patient, or the anatomy to which contrast material was supplied during the acquisition of the CT data set. The steps in the process are visually represented in FIG. 2

A fluoroscopic image of the patient is obtained (step 570). This real-time image is from the same orientation as was used to compute the virtual roadmap mask, and so the fluoroscopic images are congruent with the virtual roadmap mask images. However, the virtual roadmap mask image has suppressed the anatomical structures that were not highlighted by the contrast media. When a treatment device, such as a catheter, is introduced into the field of view of the fluoroscopic image, and is within the anatomical structure that had been highlighted by the contrast media, the catheter may be distinctly seen in the virtual roadmap image produced when the two images as superimposed, such as by subtraction of the gray-scale values (step 580).

The resultant virtual roadmap image may then be used to visualize the interaction of the catheter with the patient to facilitate diagnosis or treatment. During the course of the procedure, the fluoroscopic viewing aspect may need to be changed so as to better visualize the catheter in the patient, or the result of the treatment. When the orientation of the fluoroscopic device is changed with respect to the patient, the position of the device may be monitored by sensors or by dead-reckoning (that is, the measurement of, for example, the number of steps performed by the various stepping motors in the robotic control). This new position may be used to recomputed DRRs and virtual roadmap masks corresponding to the new position so that the virtual roadmap may continue to be used.

The use of virtual roadmap images may be discontinued at any time and fluoroscopic images alone or difference fluoroscopic images used. The user may return to the use of the virtual roadmap images at any time.

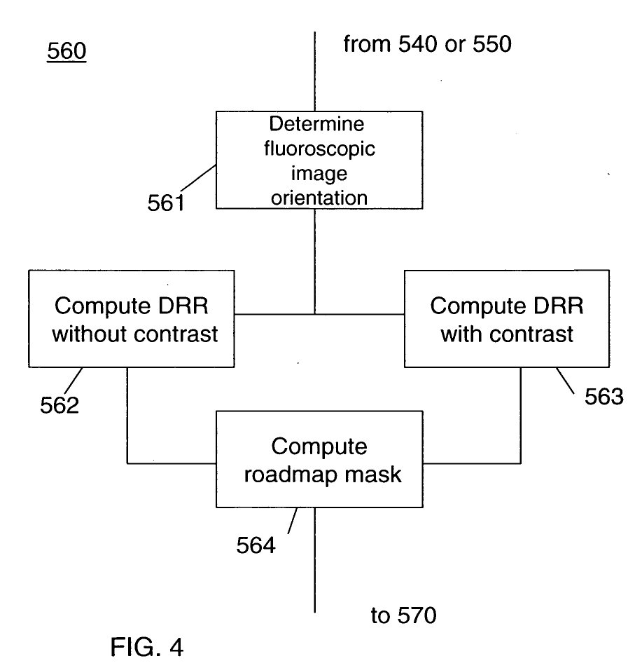

The step 560 may further include, as shown in FIG. 4, the steps of determination of the X-ray apparatus orientation (step 561), the computation of a DRR without contrast (step 562), and the computation of a DRR from the same orientation with contrast (step 563). At least one of the images produced in steps 562 and 563 may be used as a virtual roadmap mask image (step 564) and differenced with respect to the fluoroscopic image to produce the virtual roadmap image (step 580).

In an example of the use of virtual roadmap fluoroscopy, a clinical workflow for a neurological procedure is described in the treatment of a cerebral aneurysm by embolization.

An aneurysm is a weak part of an artery that, over time, has stretched and grown out outward from the artery to form a ball or pouch that blood flows in and out of. The risk associated with such an aneurysm is haemorrhage or rupture of the aneurysm wall and bleeding into the surrounding tissue resulting in damage to the brain by one of a number of physiological mechanisms.

Embolization is the process of occluding a vessel or endovascular structure. Occlusion is accomplished by deploying an embolic material (e.g. polymerizing agent, particles, reactive agent, or coils) into the vessel or lumen to be embolized.

An endovascular procedure may start with access to the endovascular system. For cerebral procedures, access is typically obtained at the femoral artery. After access, a guidewire and over-the-wire catheter are advanced from the femoral artery into the aorta and on up to the aortic arch. From the aortic arch the main cerebral arteries (left internal carotid, left vertebral, right internal carotid, and right vertebral) are accessed and diagnostic angiography performed to study the vessels and locate aneurysms. A 3D imaging scan is obtained for each artery that contains an aneurysm that appears to require treatment. The scan may be performed using a contrast injected into the artery in question, and an image may be reconstructed without mask image subtraction.

A physician may review the 3D dataset and identify the primary fluoroscopic working views that will be most beneficial when attempting to occlude the aneurysm. A first view will typically be orientated such that there is a clear delineation of the aneurysm and the vessel: a view where the plane of the aneurysm is projected as a line in the image. A second view may the physician with a full projection of the aneurysm neck, to facilitate occlusion device placement relative to the aneurysm opening.

The user may enable the virtual roadmap feature, and the system will compute virtual roadmap mask images for each of the fluoroscopy system views (imaging planes). The physician may use a combination of virtual roadmap fluoroscopy and regular fluoroscopy to navigate a catheter and guidewire pair through the parent arteries to the location of the aneurysm. The physician may then orient the system's imaging plane or planes to the working view or views identified earlier, or as become desirable during the procedure.

Digital subtraction angiography (DSA) imaging may be used to visualize blood flow into the aneurysm before treatment and to confirm that the working views are acceptable. The user may then center the micro-catheter in the neck of the aneurysm via virtual roadmap fluoroscopy. The physician may, for example, insert embolic coils into the aneurysm, until the aneurysm is packed enough that no appreciable blood flow is visible in the aneurysm on a DSA. Coil insertion may be performed using virtual roadmap fluoroscopy so that the coils are placed fully in the aneurysm and do not loop out of the aneurysm into the artery. Virtual roadmap fluoroscopy may be used at any number of additional working views, as the system automatically creates a new roadmap mask after each imaging plane adjustment. In a single plane fluoroscopic system, switching between working views may frequently occur.

Post-treatment DSA and/or CT-like images may be used to verify successful embolization of the aneurysm and that the treatment caused no damage to the parent vessels. The treatment devices may then be removed from the patient and the arterial access site closed.

The combination of hardware and software to accomplish the tasks described herein may be termed a system. The instructions for implementing processes of the system may be provided on computer-readable storage media or memories, such as a cache, buffer, RAM, removable media, hard drive or other computer readable storage media. Computer readable storage media include various types of volatile and nonvolatile storage media. The functions, acts or tasks illustrated or described herein may be executed in response to one or more sets of instructions stored in or on computer readable storage media. The functions, acts or tasks may be independent of the particular type of instruction set, storage media, processor or processing strategy and may be performed by software, hardware, integrated circuits, firmware, micro code and the like, operating alone or in combination. Some aspects of the functions, acts, or tasks may be performed by dedicated hardware, or manually by an operator.

The instructions may be stored on a removable media device for reading by local or remote systems. In other embodiments, the instructions may be stored in a remote location for transfer through a computer network, a local or wide area network, by wireless techniques, or over telephone lines. In yet other embodiments, the instructions are stored within a given computer, system, or device.

Communications between the devices, the system, subsystems, and applications may be by the use of either wired or wireless connections. Wireless communication may include, audio, radio, lightwave or other technique not requiring a physical connection between a transmitting device and a corresponding receiving device. While the communication may be described as being from a transmitter to a receiver, this does not exclude the reverse path, and a wireless communications device may include both transmitting and receiving functions.

Where the term “wireless” is used, it should be understood to encompass a transmitting and receiving apparatus, a transceiving apparatus, or the like, including any antennas, and electronic circuits for modulating or demodulating information onto an electrical signal, which may subsequently be radiated or received. The term wireless, when describing an apparatus, does not encompass an electromagnetic signal in its free-space manifestation. A wireless apparatus may include both ends of a communications circuit or only a first end of a circuit where another end of the circuit is a wireless apparatus interoperable with the wireless apparatus at the first end of the circuit. An example of such a wireless apparatus is one meeting the requirements of IEEE Standard 802.11 b/g.

A system or treatment suite may have additional treatment and diagnostic equipment such as a patient monitor, a data terminal for inputting and outputting patient data, such as demographic data, insurance card, laboratory data, patient history and diagnosis information (for example, in the form of a “wireless notebook PC” or the like), various video displays, including projection displays, for displaying data and images, and a digital camera unit for monitoring and video documentation of the individual diagnostic and therapeutic steps. Various signal and data processors may be combined as appropriate with data storage means, displays, control terminals and the like and configured by machine readable instructions to perform the functions and operations described herein.

For the purposes of this specification, the term pre-operatively may be considered to represent a time where diagnosis is being performed, including obtaining such data as electrophysical data, or angiographic data or the like, or any time preceding the treatment. During this period, the procedures may be non-invasive or minimally invasive, as is known in the art, such as the insertion of a measurement catheter or the administration of contrast agents, or the like. Intra-operatively may be considered to represent the time where a specific course of treatment is being administered, based on the pre-operative data. The course of treatment may be modified during the intra-operative procedure based on the results being obtained and other considerations. Although the data for CT-like images is usually obtained during the pre-operative period, this is due primarily to the time needed to obtain and process the data using existing commercial equipment. The distinction between the pre-operative and intra-operative periods is likely to be reduced or eliminated as processing speeds increase. As such, the terms pre-operative and intra-operative should not be considered to be disjoint time frames, as it may be come possible to obtain data for CT-like images during the treatment procedures.

While the methods disclosed herein have been described and shown with reference to particular steps performed in a particular order, it will be understood that these steps may be combined, sub-divided, or reordered to from an equivalent method without departing from the teachings of the present invention. Accordingly, unless specifically indicated herein, the order and grouping of steps is not a limitation of the present invention.

Although only a few examples of this invention have been described in detail above, those skilled in the art will readily appreciate that many modifications are possible without materially departing from the novel teachings and advantages of the invention. Accordingly, all such modifications are intended to be included within the scope of this invention as defined in the following claims.

What is claimed is:

1. A system for imaging a patient, comprising:
an imaging device; and
a digital storage device,
wherein the imaging device is configurable to collect imaging data suitable for producing CT-like images, and image data processed to form a virtual roadmap mask image which has the characteristics of a 2-dimensional radiograph taken from an orientation with respect to the patient.

2. The system of claim 1, wherein the virtual roadmap mask image is processed so as to have an inverted gray scale when compared with a fluoroscopic image.

3. The system of claim 2, wherein a fluoroscopic image of the patient taken from an orientation which is the same as the orientation used to produce the 2-dimensional radiograph is subtracted from the virtual roadmap mask image to form a virtual roadmap image.

4. The system of claim 3, wherein the orientation used to produce the virtual roadmap mask image is adjusted so as to achieve a best match with the fluoroscopic image.

5. The system of claim 1, the virtual roadmap mask image is produced for an orientation that is oblique to the axis about which the CT-like image data was obtained.

6. The system of claim 1, wherein a virtual image of a catheter is subtracted from the virtual roadmap mask image.

7. The system of claim 6, wherein the location and orientation of the catheter tip is determined by acoustic, magnetic or electromagnetic apparatus.

8. The system of claim 1, wherein the imaging device is a C-arm X-ray apparatus.

9. The system of claim 8, wherein the C-arm X-ray apparatus is mounted to a robotic device such that an orientation thereof with respect to the patient may be controlled.

10. The system of claim 9, wherein the orientation is a present orientation.

11. The system of claim 10, wherein the present orientation is communicated to an image processor.

12. The system of claim 11, wherein the image processor is configured so that image data representing a virtual roadmap mask is produced for the present orientation.

13. The system of claim 1, wherein a fluoroscopic image of the patient with a contrast agent introduced into a vessel is subtracted from the virtual roadmap mask.

14. The system of claim 1, wherein at least some of the imaging data is transmitted to, or received from, a remotely located data base over a telecommunication system.

15. The system of claim 14, wherein the data base is configured to conform to, or to be interoperable with, a DICOM (Digital Communications in Medicine) conforming system.

16. A method of imaging a patient, the method comprising:
obtaining CT-like image data of a patient;
computing a digitally reconstructed radiograph for the image data, corresponding to an orientation of a fluoroscopic apparatus;
preparing a mask image from the digitally reconstructed radiograph;
obtaining a fluoroscopic image of the patient from a same orientation as used to compute the digitally reconstructed radiograph; and,
subtracting the fluoroscopic image from the mask image.

17. The method of claim 16, further comprising displaying the image resulting from subtracting the fluoroscopic image from the mask image.

18. The method of claim 16, wherein the CT-like image data is obtained using a contrast agent.

19. The method of claim 16, wherein the orientation of the fluoroscopic apparatus is altered to a new orientation and the steps of computing, preparing, obtaining and subtracting are repeated for the new orientation.

20. The method of claim 16, wherein the CT-like data is obtained using a C-arm X-ray apparatus.

21. The method of claim 20, wherein the C-arm X-ray apparatus is used to obtain the fluoroscopic image data.

22. The method of claim 16, wherein at least a portion of the image data is retrieved from a remote data base.

23. A computer program product stored or distributed on a computer readable medium, comprising:
instructions for configuring a computer to:
receive image data suitable for production of a CT-like image of a patient;
compute a 2-dimensional radiograph using the CT-like data in a same orientation as a fluoroscopic image of the patient;
compute a virtual roadmap mask image
compute a virtual roadmap image by subtracting the virtual roadmap mask image from the fluoroscopic image.
